(19) United States
(12) Patent Application Publication
(10) Pub. No.: US 2021/0378514 A1
(43) Pub. Date: Dec. 9, 2021

(54) SMARTPHONE-BASED MENTAL HEALTH AND ADDICTION ASSESSMENT

(71) Applicants: Caden R. Moenning, Punta Gorda, FL (US); Stephen P. Moenning, Fort Myers, FL (US); John E. Acker, Irving, TX (US)

(72) Inventors: Caden R. Moenning, Punta Gorda, FL (US); Stephen P. Moenning, Fort Myers, FL (US); John E. Acker, Irving, TX (US)

(21) Appl. No.: 17/339,003

(22) Filed: Jun. 4, 2021

Related U.S. Application Data
(60) Provisional application No. 63/035,481, filed on Jun. 5, 2020.

(51) Int. Cl. A61B 5/00; G16H 50/30; G16H 40/67

(52) U.S. Cl. CPC A61B 5/0022; G16H 50/30; G16H 40/67; A61B 5/0004; A61B 5/6898

(57) ABSTRACT

Methods and systems for detecting relapse or recovery events of a user (e.g., an individual, a patient undergoing mental health and addiction treatment) are disclosed. A mobile device detects a communication from a user. The mobile device determines present attributes associated therewith. The mobile device determines, as a function of the communication and the present attributes, a likelihood that a relapse or a recovery event associated with the user is to occur. In response to determining that a relapse or recovery event is to occur, the mobile device transmits a content communication responsive to the relapse or recovery event to the user

### CROSS-REFERENCE TO RELATED APPLICATION

This application claims priority to U.S. Provisional Patent Application Ser. No. 63/035,481, filed Jun. 5, 2020.

### FIELD

The present disclosure relates to mobile communication devices, and more particularly, to performing mental health and addiction assessment using various types of user input.

### BACKGROUND

In the field of medicine, mental health and addiction may need to be assessed. Further, the COVID-19 outbreak has resulted in an escalation of mental health issues. The need for advanced mobile mental health/addiction (MH/A) assessment is of increasing importance. For example, the World Health Organization (WHO estimates 31 million persons in the world have a substance use problem. Health care professionals are concerned about an increase in substance use disorders that may parallel the mental health crisis. The recent COVID-19 pandemic and financial pressures may further exacerbate the co-occurring disorders of mental health and addiction. Mobile devices can provide insight into the mental health of an individual. For example, the current mental state of the individual can influence usage behavior of the mobile device.

### BRIEF DESCRIPTION OF THE DRAWINGS

FIG. 1 illustrates an example computing environment in which an application executing on a mobile device performs mental health and addiction management based on user inputs, according to an embodiment.

FIG. 2 further illustrates a block diagram of the mobile device of FIG. 1, according to an embodiment.

FIG. 3 illustrates a block diagram of an environment of the application of FIG. 1 in execution, according to an embodiment.

FIG. 4 illustrates a flow diagram of a method for training an individualized model for a user to detect addiction recovery or relapse events, according to an embodiment.

FIG. 5 illustrates a flow diagram of a method for detecting and treating addiction recovery or relapse events, according to an embodiment.

### DESCRIPTION OF THE ILLUSTRATIVE EMBODIMENTS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

Embodiments presented herein disclose techniques for assessing mental health of an individual based on various user inputs on a device. More particularly, the techniques disclose a mobile device application that evaluates user input on the mobile device to detect instances in which a user, such as a mental health or an addiction patient, is undergoing a mental health event, such as indicators that the user is progressing through recovery or approaching an addiction relapse. For example, the mobile application may integrate with communication applications (e.g., messaging applications, voice chat applications, video-based communication applications, etc.) to obtain usage behavior by the user of the mobile device. In doing so, the mobile application may evaluate the user input provided by such communication applications, such as text messages received by or sent to the user, voice communications, facial expressions captured by a camera of the mobile device, and the like, using various techniques, such as against machine learning models, to identify whether the user input corresponds to some recovery or relapse event. If so detected, the mobile application may carry out a number of treatment actions, such as transmitting content to the user for providing reassurance, validation, and the like, to reinforce recovery behavior or mitigate behavior indicative of a potential relapse.

Advantageously, such an approach provides an additional mode of treatment for a user compared to current approaches which require manual or impersonalized intervention. Further, the described approach uses patient associated events as the input data and as a trigger for the data. In some embodiments, the approach converts patient-associated events to a workable integer. Other advantages include the concept of gathering the associated events of a patient as data input, trigger and the affected output, randomizing and anonymizing the patient associated data before the application is active, independently placing patient associated data in a sequence of serially structured databases, conversion of any unstructured text into numbers, using Natural Language Processing and Machine Learning Algorithms to serially applied databases, the construct of these events taking place in a given order, creating a system and method of gathering this unstructured data and processing it from memories and emotions into a simple integer, having that analysis represent a polarity of mental health or addiction situations, and a reduction of the analysis down to a binary state of recurrence or remission.

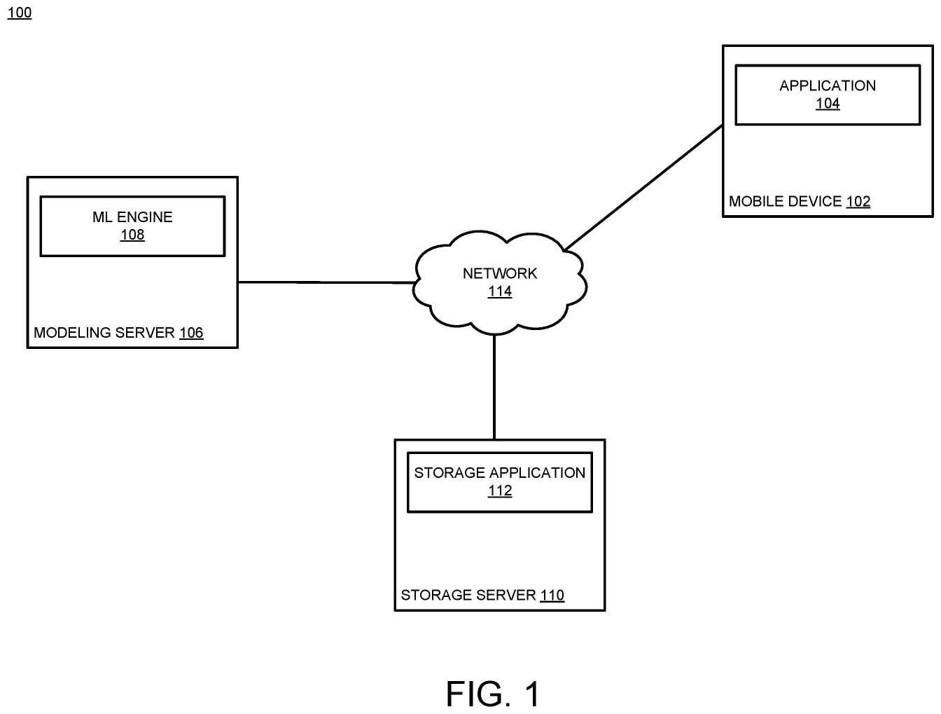

Referring now to FIG. 1, an example computing environment 100 is shown. Illustratively, the computing environment 100 includes a mobile device 102, modeling server 106, and a storage server 110, each interconnected with a network 114 (e.g., a local area network, a wide area network, the Internet, etc.). The mobile device 102 may be embodied as any portable computing device operable by a user, such as a smartphone, tablet computer, wearable device, and so on. The modeling server 106 and the storage server 110 may be embodied as a physical computing device (e.g., a desktop computer, laptop computer, workstation, etc.) or a virtual computing instance executing in the cloud.

The mobile device 102 includes an application 104 for mental health assessment and addiction recovery. The application 104 integrates with communication applications and functionalities within the mobile device 102 to detect, within various user inputs, instances corresponding to recovery and relapse events of a user, e.g., an individual undergoing treatment for mental health issues, substance abuse addiction, and the like. User inputs can include, for example, incoming text messages from third parties, outgoing text messages, voice communications to or from third parties, facial expressions communicated via video communications, and the like. To detect recovery or relapse events, the application 104 may evaluate the underlying content of the communications for terms, behavior, or patterns that correspond to such events. In response to detecting such events, the application 104 may cause the mobile device 102 to transmit communications to reinforce or deescalate the detected behavior.

In an embodiment, the application 104 may use a variety of machine learning and artificial intelligence techniques to detect recovery or relapse events. For example, the application 104 may include one or more machine learning (ML) models trained to detect such events. The application 104 may further train the machine learning models based on the user inputs to further individualize the machine learning models for the user. As another example, the application 104 may use natural language processing (NLP) to evaluate text inputs (or voice-to-text inputs) against a corpus of mental health and addiction indicators to determine a likelihood of a communication corresponding to a recovery or relapse event. Such corpus may be embodied in one or more databases further described herein.

The techniques performed by the application 104 may be self-contained within the mobile device 102. Of course, other embodiments may include the use of remote computing systems to assist with various functionalities of the application 104. For example, the modeling server 106 may include a ML engine 108 to assist the application 104 in training and retraining ML models used in detecting recovery and relapse events. In addition, the storage server 110 may include a storage application 112 used to store terms provided by the aforementioned databases. In an embodiment, the application 104 may retrieve data associated with a patient user from one or more databases, in which the data in the databases are analyzed using a variety of NLP and ML algorithms (e.g., subjective key texting, serial database positioning for text mining/machine learning algorithms, conversion of texting emotions to simple integers, etc.).

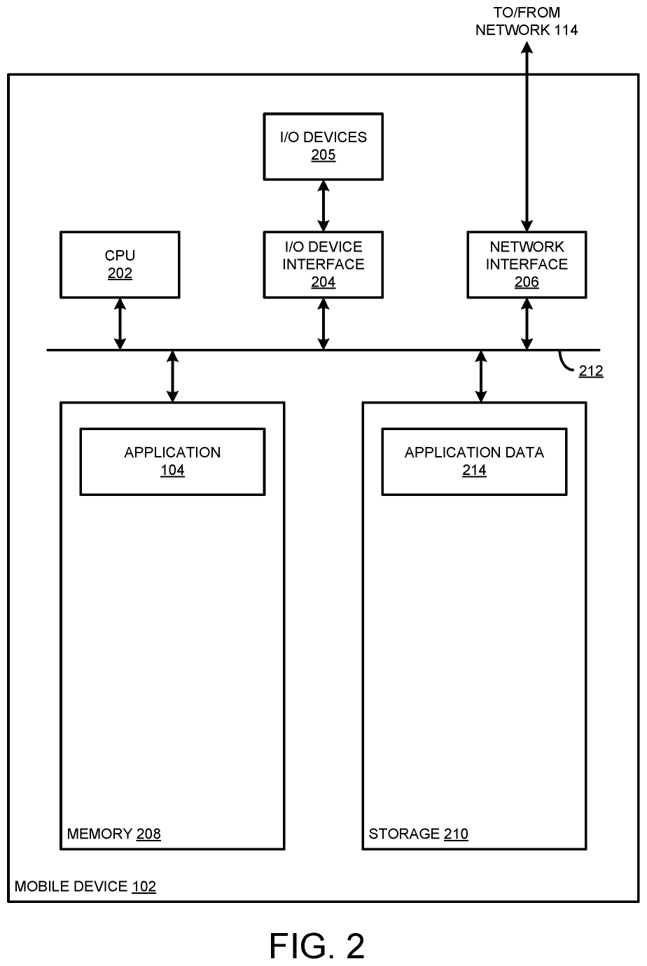

Referring now to FIG. 2, the mobile device 102 is further shown. Illustratively, the mobile device 102 includes, without limitation, a central processing unit (CPU) 202, an I/O device interface 204, a network interface 206, a memory 208, and a storage 220, each interconnected via an interconnect bus 212. Note, CPU 202 is included to be representative of a single CPU, multiple CPUs, a single CPU having multiple processing cores, and the like. Memory 208 is generally included to be representative of a random access memory. Storage 210 may be a disk drive storage device. Although shown as a single unit, storage 210 may be a combination of fixed and/or removable storage devices, such as fixed disc drives, removable memory cards, or optical storage, network attached storage (NAS), or a storage area network (SAN). The 110 device interface 204 may provide a communications interface with 110 devices 205 (e.g., an image capturing device, speaker, microphone, a biometric data capturing device, etc.), enabling the mobile device 102 to capture, for example, self-taken images of a user's facial expressions from a front-facing camera thereof. The network interface 106 may be embodied as any hardware or circuitry to enable the mobile device 102 to communicate over the network 114.

Illustratively, the memory 208 includes an application 104, which may be embodied as program code to be executed on the CPU 102 to perform the claimed functions further described herein. The storage 210 may include application data 111 used for the application 104. For example, the data 111 may be organized within a data store of the storage 210, such as a content database and a control database, both of which are further described herein. Application data 111 also pertains to configuration data for the application 104 as well as other application data further described herein.

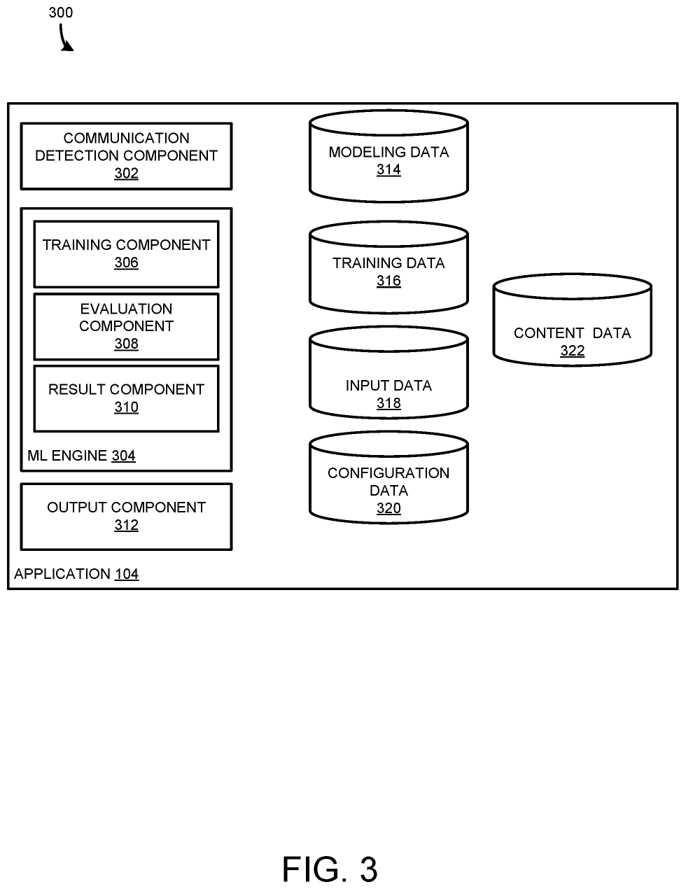

Referring now to FIG. 3, the application 104 may provide an environment 300 for carrying out functions described herein, including training one or more ML models for detecting recovery or relapse events of a user, detecting, by a mobile device, an incoming communication to or outgoing communication from the user, determining present attributes associated with the mobile device 102, determining whether a relapse or recovery event is occurring based on the communication and present attributes, and upon such a determination, transmitting content to the user responsive to the event. As shown, the environment 300 includes a communication detection component 302, a ML engine 304, and an output component 312. The environment 300 may include and/or manage modeling data 314, training data 316, input data 318, configuration data 320, and content data 322.

Modeling data 314 may be embodied as one or more ML models for detecting recovery or relapse events. One example of a ML model included with the modeling data 314 is a natural language processing (NLP) model that recognizes text leading to addiction recovery or relapse for a generic individual patient. Training and validating such a model may include a variety techniques, such as Bidirectional Encoder Representations from Transformers (BERT), sentiment analysis, supervised or unsupervised machine learning for verbiage related to addiction, relapse, and recovery. As another example, an analysis of discourse positions provides a mode for linking texts to known mental health and addiction social situations. Conversation analysis allows user input to be used to define additional social situations. Critical discourse analysis allows the application 104 to identify critical social situations in user input, e.g., by identifying, in the user input, critical social indicators such as the term “suicide” or “heroin.” Content analysis allows the application 104 to develop analytical categories for constructing a coding frame to be applied to user input. Foucauldian intertextuality allows the application 104 to compare user input similarities and differences between outgoing data points with respect to implicit presuppositions recognized by comparing new outgoing communications to historical communications found either in a storage of the mobile device or remote storage databases. In addition, the application 104 may use lexical resources (e.g., lexical databases or publicly available lexical resources) to identify terms in common parlance. The application 104 may also use Linguistic Inquiry and Word Count (LIWC) to group outgoing communications into categories, classes, and simple words. As stated, the application 104 also uses language models for probabilistic representation of natural language being used as a predictive model of subsequently outgoing communications. Such a model can be helpful as a predictor of a social outcome of remission or recurrence even if the user provides “code” words in the communications (e.g., using “hillbilly heroin” as a slang term for the drug Adderall). Using a language model, the application 104 may identify a term, such as “cocaine” and determine a probability of finding another term such as “crack” in a given proximity. The application 104 may use supervised learning techniques, decision trees, neural networks, support vector machines (SVM) to learn from a history of outgoing text events of natural and slang text that the user communicates in a social situation. The application 104 may also use thematic analysis to identify and report patterns of contextual themes found within a group of text (e.g., outgoing texts consistent with addiction remission or recurrence). Various software can also be integrated with the application 104, such as computer-assisted qualitative data analysis software (CAQDAS), to improve search techniques when text mining is used. Other examples of NLP and text analysis that the application 104 may use are narrative analysis, metaphor analysis, word and text relatedness, text classification, sentiment analysis, and topic models.

The modeling data 314 may also include a personalized model for a user of the application 104. For instance, the model may be individualized by configuring and then training into the generic individual patient model based on obtained input data, such as verbiage (e.g., words, phrases, words in proximity, words with sentiment, etc.) used by the user or a user contact pertaining to addiction recovery or relapse, GPS area locations related to relapse or recovery for the individual for geo-fencing, contact information associated with other individuals that might communicate with the user via mobile device regarding recovery or relapse events, and the like. In addition, a variety of generic machine learning models may also be adapted, such as multiple models to specific addiction types (e.g., gambling, drug abuse, alcohol abuse, etc.).

Training data 316 may embodied as any data used to train the ML models of the modeling data 314 discussed above. The training data 316 can include the inputs discussed above, such as for use in training the individualized patient model. Input data 318 may be embodied as any data obtained based on usage behavior by the user, such as text data, voice data, audio data, image data, and the like. Configuration data 320 may include any settings or specifications for tuning the ML models of modeling data 314 (e.g., retraining frequency, timer periods, whether to enable or disable remote model retraining, and the like).

The content data 322 (also referred to herein as “text-to-self” (TTS) data) may be indicative of content that the application 104 may transmit to the user via a variety of communication mechanisms (e.g., text messaging, audio messaging, email, and the like) to provide positive reinforcement to the user in response to a detected recovery or relapse event. The content data 322 may include generic phrases and recordings (text content reading “I am proud of you” or “Keep going”) or content personalized for the user (e.g., audio messages recorded by a counselor or therapist for the user).

The communication detection component 302 may be embodied as any device, software, or circuitry for obtaining and further processing communication data, such as text messaging data, video data, audio data, and the like, transmitted by or received by the mobile device 102 (e.g., via various communication applications executing thereon). The communication detection component 302 may associate the detected communication data with timestamp information indicative of when the communication was received or transmitted. The communication detection component 302 may then transmit the communication data to the ML engine 304.

The ML engine 304 may use various machine learning techniques to train and re-train the modeling data 314 and detect recovery and relapse events. As shown, the ML engine 304 further includes a training component 306, an evaluation component 308, and a result component 310. The training component 306 may initialize a machine learning model and retrain the model based on new inputs. The evaluation component 308 may perform ML techniques on input data 318 received from the user. For example, the evaluation component 308 may perform natural language processing (NLP) techniques (e.g., BERT, sentiment analysis, supervised or unsupervised techniques, etc.) to identify content and characteristics associated with the communication. The evaluation component 308 may apply the data to the models to determine whether the input data corresponds to a recovery or relapse event. The result component 310 receives the output from the evaluation component 308 and may transmit the result to the output component 312.

The output component 312 may be embodied as any device, circuitry, or software to evaluate the result provided by the ML engine 304 and determine whether to perform an action in response. For example, the output component 312 may perform an action in response to output indicating that a communication sent by a user is indicative of a recovery or relapse event. The output component 312, in doing so, may further determine appropriate content data 322 to send to the user.

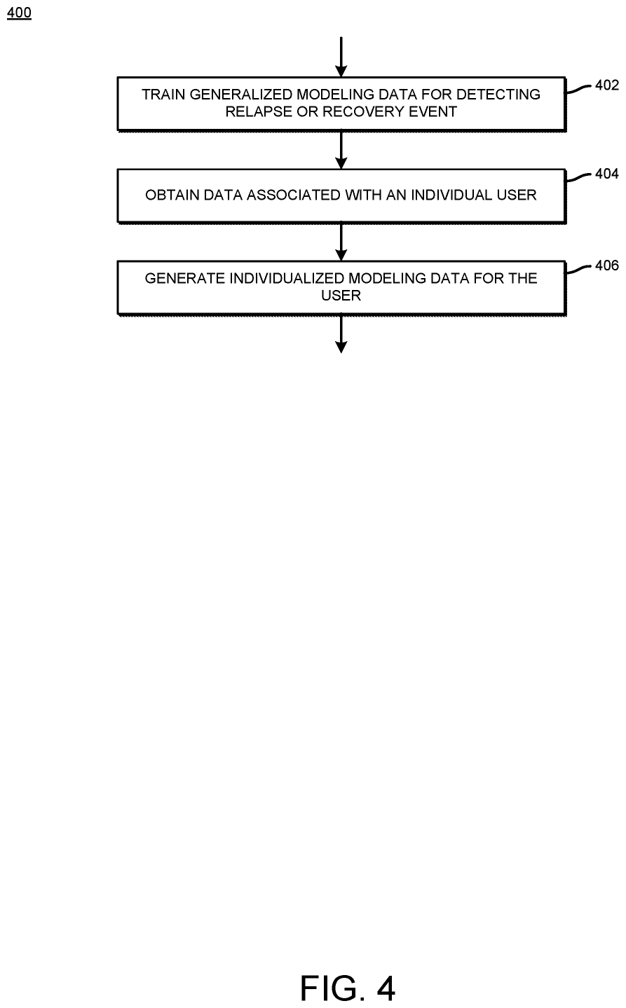

Referring now to FIG. 4, the mobile device 102, in operation, may perform a method for generating a machine learning model for mental health assessment and addiction recovery. As shown, the method 400 begins in block 402, in which the mobile device 102 (via the application 104) trains generalized modeling data for detecting a recovery or relapse event. Particularly, the application 104 may build a NLP model that recognizes text leading to addiction recovery or relapse for a generic addict patient. As stated, the training and validation may include techniques such as BERT, sentiment analysis, supervised or unsupervised machine learning, and the like. The application 104 may re-train the generic model using a variety of additional information (e.g., generalized data from a plurality of patients in the anonymized aggregate). For example, the application 104 may retrain the model a specified amount of times per a given period by combining a collection of each of the latest versions of the individual models as input, so long as enough active individual models exist to make the collection representative of a generic patient.

The generated machine learning model serves as a starting point for an individual patient model. In block 404, the mobile device 102 (via the application 104) obtains data associated with the user. The data serves as a patient-specific configuration for a subsequently generated model. The obtained data can include verbiage (e.g., words, phrases, words in proximity, words with sentiment, etc.) used by the user or an associated contact pertaining to the addiction recovery or relapse of the user. The obtained data can also include GPS area locations relating to a relapse or recovery of the individual (e.g., determined based on an assessment by a professional). Such GPS data can be used by the application 104 to establish a geo-fence for determining a likelihood that a user is undergoing a recovery or relapse based on location. The obtained data can further include contact information (including relationship to the user), which can be indicative of a person that the user may communicate with in a recovery or relapse event. In block 406, the mobile device 102 (via the application 104) generates an individualized model for the user using the obtained data as input.

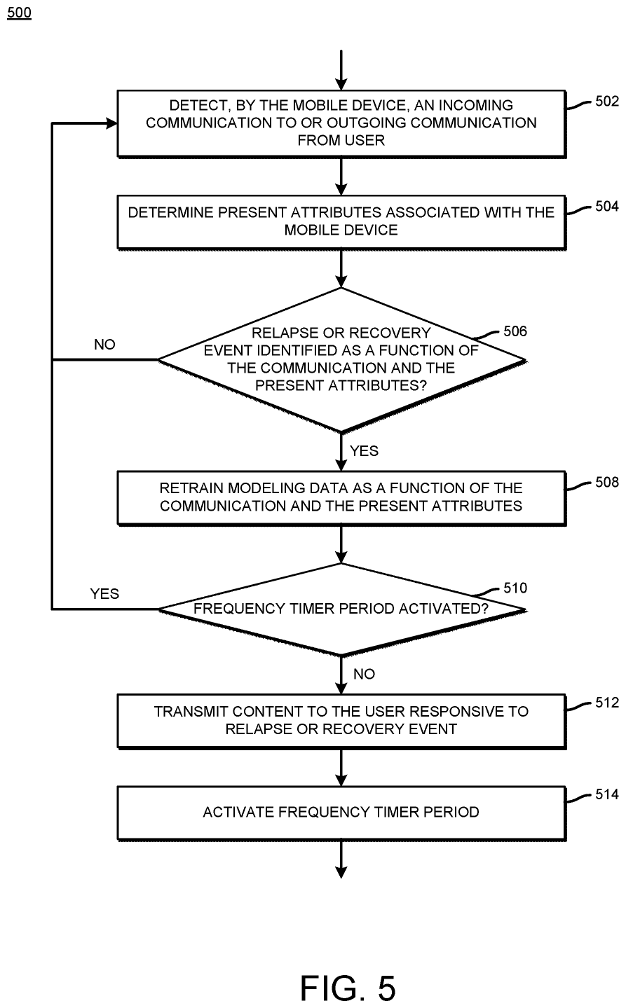

Referring now to FIG. 5, the mobile device 102, in operation, performs a method 500 for detecting and treating addiction recovery or relapse events. As shown, the method 500 begins in block 502, in which the mobile device 102 (via the application 104) detects an incoming communication to or outgoing communication from the user. For example, the communication can be a text message, a voice or phone call communication, a video chat session, and so on. Further, in block 504, the mobile device 102 (application 104) determines present attributes associated therewith. Such attributes include GPS location data, a date and time information, and the like. The application 104 may correlate such attributes and communications with a relapse or recovery event. In block 506, the mobile device 102 determines whether a relapse or recovery event is likely occurring as a function of the communication and the observed present attributes. For example, the application 104 may identify anomalies associated with a time of day in which the user sends a text message to a given contact. As another example, the application 104 may observe tendencies associated with a given location that relate to an addiction relapse. If the mobile device 102 does not identify such event, the method 500 returns to block 502.

However, if the mobile device 102 identifies a recovery or relapse event, then in block 508, the mobile device 102 (via the application 104) retrains the machine learning models as a function of the communication and the present attributes. For example, if the event actually takes place, the application 104 may use, for retraining on the individual model, an amount of minutes of communication data and GPS data prior to the date and time of the actual relapse event. Doing so allows the individual-specific model to improve effectiveness of detection of an event for that individual, factoring in the individual's changes in significant vocabulary and GPS areas and contacts, even in situations in which the configuration of the application 104 is not regularly modified to track such changes. As another example, the application 104 may use, for retraining the model, an amount of minutes of communication data and GPS data prior to the event, as well as any data indicating a trigger of the event (e.g., text communications correlating to a subsequent relapse or a recovery event). The models may weight the triggering data, and in retraining, the application 104 may reweight based on additionally observed triggering data.

In block 510, the mobile device 102 (via the application 104) may determine whether a frequency timer is activated. In an embodiment, the application 104 may activate a frequency timer of a specified period (e.g., 15 seconds, 30 seconds, 5 minute, etc.) to limit the amount of content transmitted to the user by the application 104. If a frequency timer is currently activated, then the method 500 returns to block 502. Otherwise, in block 512, the mobile device 102 (via the application 104) transmits content to the user responsive to the detected relapse or recovery event. To do so, the application 104 may retrieve content responsive to the type of event. The content may be in various formats, such as audio recordings, video recordings, text messages, and the like. In block 514, the mobile device 102 (via the application 104) may activate or reset the frequency timer period.

To illustrate the aspects of the aforementioned disclosure, consider the text conversation detailed in the below tables between a patient user “Paul” and a drug dealer “Archie.”

TABLE 1
Paul's conversation

| Type of Thought Reaction Outgoing Text | Recurrence (−)/Remission (+) Score |
|---|---|
| Instinctive Reaction | −1 ‘ox’ is recognized as oxycodone and associated with |
| ‘If I don't get some Ox [street slang for | recurrence in the database |
| oxycodone], I am going to bust a gut!’ | |
| Learned Reaction | −1 ‘Archie’ is used in close proximity to ‘ox’ both words |
| ‘Archie needs to help me’ | are associated with recurrence |
| Deliberative Reaction | +1 ‘hold off’ is registered as a remission phrase and when |
| ‘maybe I can hold off- I don't need to see him’ | used in close proximity to ‘Archie’ |
| Reflective Thinking | +1 ‘hold off’ remission phrase used twice in close |
| ‘I think it is best I hold off’ | proximity |
| Self-Reflective Thinking | +1 Application recognizes ‘I know’ phrase as self- |
| ‘I know it could make me mad if I used again’ | reflective remission thinking |
| Self-Conscious Emotion | +1 Application recognizes ‘Archie’ as a key name of |
| ‘Archie could understand’ | individual in Paul's addiction. ‘Archie (Name)could |
| | understand’ as self-conscious emotion of remission not |
| | just a statement about a guy named ‘Archie’ |

In the first column, Paul's conversation is a one-way, outgoing texting corpus involving elemental text. These outgoing texts are processed by the application 104. In the conversation above, artificial intelligence is used for word recognition via a customized lexical database, processed through the application 104 using word proximity, sentiment analysis and subjectivity analysis to generate a coordinate score for Paul's text transaction of (−2, +4). Improving upon prior art, current ‘coordinates’ described above are derived independent of a network connection. The application 104 has taken a text stream of patient texts and generated the texts into a quantifiable score for the patient mental health and addiction assessment. This one text stream has scored as follows: −1, −1, +1, +1, +1, +1. The application 104 may sum the individual integers to further refine and store the text strings as an overall ‘good’ outcome for the texting moment as demonstrated by an over-all ‘net positive’ coordinate of (−2, +4).

Continuing the present example, because the key phrases ‘I don't need to see him’ and ‘I hold off’ are in proximity in the content data 322. These phrases may serve as a trigger to send a content communication from the chosen imprinter, which pertains to a person of significance in the life of the subject. The application 104 may immediately send a text to self with an audio file embedded therein, in which the content is recorded by his guidance counselor whom he has chosen as his imprinter. This audio file may be a “Text To Self” relaying to him a congratulatory statement in her voice, “I am proud of you Paul.” Paul scores another+5 for the positive content communication. At the end of the time it took to send a text exchange Paul has scored co-ordinates of (−2, +4, +5) for that exchange.

| Type of Thought Reaction | Coordinate Score | Content Generated | Content Sent |
|---|---|---|---|
| Outgoing Text | | | |
| Instinctive Reaction | −1 | | |
| ‘If I don't get some Ox (street slang | | | |
| for oxycodone), I am going to bust | | | |
| a gut!’ | | | |
| Learned Reaction | −1 | | |
| ‘Archie needs to help me’ | | | |
| Deliberative Reaction | +1 | Activated via ‘I don't | Therapist voice is |
| ‘maybe I can hold off- I don't need | | need him’ | Activated to send |
| to see him’ | | | ‘I am proud of you |
| | | | Paul |
| Reflective Thinking | +1 | Activated via phrase | |
| ‘I think it is best I hold off’ | | ‘I hold off’ | |
| Self-Reflective Thinking | +1 | | |
| ‘I know it could make me mad if I | | | |
| used again’ | | | |
| Self-Conscious Emotion | +1 | | |
| ‘Archie could understand’ | | | |
| Subtotal score | (−2, +4) | | +5 |
| Total coordinate score | (−2, +4, +5) = +7 | | |

The patient may keep the ‘key texting’ data or delete the data. In an embodiment, the above text mining techniques are structured to develop rules that may help us predict what is likely to happen if a condition exists. In this way, the outgoing text have been processed (e.g., using machine learning algorithms) to predict what may happen with the addiction/mental health issue.

An embodiment disclosed herein includes a device and method of using both texting and facial analysis to reflect these states. This proposed embodiment uses a device and method combining the neuropsychological aspects of texting with facial analytics under the direction of a user. These patients associated facial fiducial points may be created outside of the application and smart phone. Having been inserted into the application, these facial data points may be retained, and the patient's picture may be deleted. Further, under the skin of the face lays some of the smallest muscles in the human body. To those people dealing with facial biometrics, these small muscles are called facial action units. When a subject processes cortical information (i.e. writing a text) this information may be associated with a facial expression. As discussed herein, unique brain waves are associated with texting. Functional MRI studies have confirmed a specific area of the brain used to process the facial expressions of emotion. Using probability algorithms, the application 104 may be able to reliably predict which emotion is associated with a particular texting output. Connecting the emotion to the facial expression is the outgoing text. The text may be indicative of a tangible manifestation of the emotion. For instance, an outgoing text stating, “I am 2 months clean of heroin” may be associated with the basic emotion of joy. Further, confidentiality may be maintained via deletion of the picture. As the picture is deleted the application 104 may retain the ‘facial grid’ and associated fiducial points.

The application 104 may also use artificial intelligence techniques to input data. Current mental health assessment techniques use an ongoing therapist-patient interaction to assess the changes in human emotional association. Existing applications are controlled via a remote backend server and requires a network typically require the subject to view a lesson module via a network. The application 104 described herein, using text mining and natural language processing, is able to be operated independent of a remote server and a network connection. Of course, the application 104 may transmit data (e.g., user input data for model training or retraining) to a remote server via a network connection to alleviate processing resources of the mobile device 102. Using the approach described herein, the application 104 may use a baseline texting database store on the mobile device 102 for addiction and mental health issues.

Generally, the application 104 detects recovery or relapse events based on patient word associations, from which preexisting approaches are not designed to interpret and quantify different emotional levels associated with persons with mental health issues and addiction. If the persons emotional state is a reflective thought or a learned reaction, the application 104, as described herein, may use texting techniques to identify, interpret, update the database, store and score the learned reaction. Further, the application 104 may quantify ecological momentary assessment of mental health and addition using an integer. Each person has many emotional and intellectual variables that define the momentary mental health and addiction state. Such states may be represented in spontaneous texting. Further, these texting words expressed as a function of time are significant and may change in context, spelling, and thematic meaning. Active texting may present words that are modified in the social environment and as such, may need complex texting techniques for analysis.

For example, assume as user of the application 104 is being treated for a heroin addiction and is new to remission (e.g., the user has been clean of heroin for two weeks). On the way to a meeting, further assume that the user passes the neighborhood where he would obtain heroin. This sight serves as a trigger. The user may then text his sponsor, “I am SOOOOO close to using.” Using the techniques described herein, the application 104 may interpret and quantify the word “SOOOOO” as significant because of proximity to the word “using.” Additionally, because of the five extra “O” characters in all caps, the techniques identify this as a “non-word” and process it through its “non-word” algorithm. In real time, using word relatedness, thematic analysis and sentiment analysis the application 104 may recognize this one phrase as a potential addictive moment. This phrase may have previously been embedded in the content database. The sensors and triggers in the context database may send an immediate content communication to the user. The content communication may be a voice file from his significant other telling the the user “stay strong, remember I Love you.” If action is taken from a recognized outgoing text, the text may be moved to an outgoing database store (e.g., on the device 102 or on a storage server). The application 104 may enable the outgoing communication be quantified as an integer which is further processed as positive or negative relative to the recovery or relapse event.

An imprimer can be defined as a person to whom another becomes attached. Attachment learning is strengthened by messaging from an individual's imprimers, from people who mean something to the individual on an emotional level. The elevated status of an imprimer is in contrast to strangers who send us a message of congratulations or a reproach to a contemplated action. For instance, if a person with mental health and severe alcohol use disorder issues texts “I am so nervous, I need a drink!” the application 104 may identify the word “nervous” in proximity to the word “need” and “drink.” The application 104 may identify these texts using the techniques described and generate a content communication from a predefined imprimer of the user. The content communication may include a video and audio file from an imprimer resulting in a strong, emotional, ecological momentary interference of the addictive moment, before it the drink occurs. In this way, application 104 content communication acts as a real-time, real-world, ‘attachment-learned’ reinforcement of the goal to not drink. Current art uses back end delivered real world assessment and smartphone delivered web-based tutorials.

A content communication serves to modify behavior in acceptable texting methods at the same time as the mental health/addiction issue. The content communication may serve to reinforce a positive experience via voice, video or textual files from an imprimer of the patient with mental health and addiction issues. The content communication may serve to censure a negative experience serving to discourage a patient approach or method via an imprimer data response. The content communication may also be defined as using the following: ‘attachment praise’—when an imprimer praises, with the intent is to elevate the action or text and ‘attachment censure’—when an imprimer scolds, with the intent to devalue the action or text.

The present disclosure overcomes the foregoing and other shortcomings and drawbacks of mental health/addiction applications. While the disclosure may be described in association with certain embodiments, it may be understood the disclosure is not limited to these embodiments. On the contrary, the disclosure includes all alternatives, modifications and equivalents as may be included within the spirit and scope of the present disclosure.

In an embodiment, the content data 322 comprises one or more databases, such as a control text database and a content communication database. These databases follow a three step pattern of data gathering, data processing and data analysis. Unstructured mental health and addiction data may be gathered and placed within the databases. These databases may utilize text mining techniques to process and prepare the data prior to analysis. Once data has been processed it may be analyzed by machine learning algorithms. The algorithm may result in a simple to understand integer with a positive or negative value. The magnitude of the final integer may reflect the intensity of the MH/A moment. The situational polarity of recovery or relapse may be determined by the attachment of a ‘+’ (positive) value or a ‘-’ (negative) value for recovery or relapse, respectfully. The first step of ‘gathering’ the data begins with the manual identification of ‘key’ people, places and things serving as a ‘trigger’ of the mental health/addiction issue. Once identified these keywords may be placed in an internal file source, the control database. The control database be embodied as a spreadsheet or a comma separated value (CSV) file. The therapist may create a 1-2 month ‘pre-trial window’ where the patient may use the prior period of time (i.e. 2 months) to gather ‘historical’ key texts are demonstrated to be associated with that patient, placed within the ‘control’ database. For example, in the prior two months, who were the key mental health/addiction people, places, and things in the life of the user.

In addition to the individual people, places and things, the key text trigger may include individual texts or phrases of emotionality reflecting a polarity state of the mental health/addiction issue. The application 104 is activated when the outgoing text is gathered from the communication device transmission text bar on the mobile device 102. The applications may then copy the outgoing text. The copy of the ‘outgoing’ text is compared to the corpora of text found in the control database. Data contained in the control database serves as a trigger for further action if a match is determined. The application 104 may use external sources such as WordNet to create a relatively robust control database.

Prior to insertion of ‘key’ texts into the ‘control’ database, all ‘key’ texts may undergo randomization and placed within a randomization database. As the table below depicts, each text is associated with a distinct integer and a positive or negative status:

| | | | Random | | |
|---|---|---|---|---|---|
| Randomized | | | Number | Integer | |
| Text | Context | Integer Value | Result | Status | Situation |
| Archie | Drug Dealer | Negative | 200 | −200 | Relapse |
| Tanya | NA Sponsor | Positive | 198 | +198 | Recovery |
| Convenience | Location to | Negative | 41 | −041 | Relapse |
| Store | buy drugs | | | | |
| “I'm not | Positive | Positive | 222 | +222 | Recovery |
| going to use” | sentiment | | | | |
| “I'm going to | Negative | Negative | 412 | −412 | Relapse |
| use” | sentiment | | | | |

The positive/negative value is correlated to “+” pertains to a recovery associated word and “−” pertains to a relapse associated word. This step occurs as the application is constructed in the therapist office, even before it is installed. It serves to prioritize anonymity and encryption before the application goes “live.” The numbers are randomly generated and do not reflect the magnitude of a relapse or recovery.

In the embodiment, the application 104 may determine “like” or “not like” or a “match” or “no match” status of the “outgoing live” text to the corresponding embedded control integer associated with that outgoing text. When the application determines a match to have occurred between outgoing text and the control text in the control database trigger is activated. Within this database, the application may associate the ‘outgoing’ text to a previously randomized distinct integer. If a match is determined to have occurred, the application determines the MH/A situational status of Relapse or Recovery.

If the match is associated with an improved mental health state or addiction state it is associated with a positive integer and recovery. A match between a negative text and a negative control text could generate a negative integer or relapse. In addition to NLP, the application 104 may use text mining techniques such as lexical resources either constructed manually or automatically, to assign positive or negative integer value to the text.

The gathered data maybe managed via a standard data managing system such as Postgres, MongoDB, or MySQL. Manually constructed algorithms maybe used as an alternative. The text analysis gathering techniques mentioned above may be used to extract, classify and form the input for machine learning. The output may give a quantifiable number reflecting a mental health and addiction state of remission (positive state) or recurrence (negative state) or neutral. Before the application produces a declaration of remission/recurrence the gathered data may be prepared and processed through the content communication database.

The second database is known as the content communication database. The second database is critical as it contains texts reflecting social or situational proximity to a recovery or relapse of the mental health or addiction issue. The machine learning algorithms may extract and determine similarities and differences between the control database and the content communication database files. With time, continued use of the application 104 and subsequent texting events, the application 104 may automatically update the control database content with new and updated patient associated data points.

If a match is determined to have occurred, it may also be associated with a positive or negative value. If the match is associated with an positive mental health state it is determined to be a state of recovery. The improved state is associated with a positive integer. A negative match between a negative control text and a negative critical content communication could generate a negative integer and be associated with a relapse situation. Incorporating steps mentioned above the application uses previously designated positive or negative integer status associated with the critical content communication. In an embodiment, the application 104 databases are internally controlled and independent of a remote server. Of course, the application 104 may store data (e.g., content communication data and historical user input) to a remote server via a network connection to alleviate processing and storage resources of the mobile device 102. Future text updates may be placed in an updated content communication database and updating the new integer as well. The new integer is linked numerically to the original integer via addition of a one tenth unit to the end of the number. For instance 100 changes to a 100.1 updated number.

Data analysis using machine learning classifiers may have four components. First, a Naïve Bayes classifier may distinguish between unstructured texts that are subjective or objective. It may use both lexical and contextual features and may be automatically generated from unannotated data using rule-based classifiers. Second, the analysis may involve identifying speech events and direct subjective expression classification. An example of a ‘speech event’ could be a text such as ‘tell you’ or ‘he said’. A subjective expression text example could be ‘am happy’ or ‘am sad’. These text examples could be identified in preparation for step three of data analysis. The third step of the data analysis is combining a Conditional Random Field Sequence tagging model along with extraction pattern learning to identify the sources of speech events and direct subjective expressions. For instance, in the text sequence reflecting a person with depression, ‘Bro, I tell you I am feeling bad.’ the source of the text maybe Paul the soldier with a history of suicidal ideations. The speech event is ‘I tell’ and the emotion event is ‘am feeling bad’. The fourth classifier is the sentiment expression classification. This uses two classifiers to find words with phrases that express positive or negative sentiments. This could include sentiment textual phrases that describe emotion like ‘I feel’ in the following text grouping: ‘I feel I am going into a dark place’. The second classifier is the identifier of polarity of emotion, like ‘dark’ symbolic within the field of mental health of a negative emotion.

An example of the trained opinion annotated corpora of text is discussed in the following. Two reference words such as ‘Archie’ (name of the patient's dealer, he has a negative connotation) and ‘Jerry’ (a ‘narcotics anonymous’ sponsor's name, he has a positive connotation) are used in this example. The application 104 calculates the polarity of a word by measuring the pointwise mutual information with the positive reference (“Jerry” to “sponsor”) and the pointwise mutual information with the negative reference (“Archie” to “drug dealer”). Pointwise mutational information is defined as the probability of seeing two words like “Jerry” (good) and “Archie” (bad) together, divided by the probability of seeing each individual name. If the names are represented as w1 (Jerry) and w2 (Archie):

PMI(w1,w2)=p(w1,w2)/p(w1)p(w2)

Using this method, a polarity score expressed as pointwise mutational information may automatically annotate the polarity of a patient with a mental health and addiction issue. Using this method a patient maybe texting their unstructured thoughts about such issues and in the background, the application 104 is processing the recent text, using pointwise mutational information to determine sentiment of the outgoing text. The application 104 may process the sentiment text trigger into a random integer, confirm the situation as relapse or recovery. Once determined as a situational context of ‘relapse’ or ‘recovery’, the content communication may be immediately sent to the user. In this example using “Archie,” the user may immediately receive a voice file stating, “you know to stay away from him!.”

Another embodiment involves the application 104 combining the texting content with the simultaneous smartphone camera capture of the user's image. In this approach, the application 104 is able to quantify emotional pictures and result in a positive or negative integer to be stored in the app that could be helpful in guiding addiction or mental health assessment. The construct of the application follows the same flow as mentioned previously.

A control facial database is created by placing the patient in a therapist directed photo studio setting. The therapist may have indirect lighting and a camera set up to record the facial photos of the patient. These facial photos may be obtained at the peak emotional moment when subject is read, a vignette designed to elicit one of six basic emotions (happy, sad, fear, anger, disgust, surprise) or twenty-one subordinate emotions. In an embodiment, the application 104 is able to quantify both the texting and/or emotional pictures and result in a positive or negative integer to be stored in the app that could be helpful in guiding addiction or mental health therapy.

The application 104 may use the control key text database as an actuator of the smartphone camera, enabling the capture of the self-image as the text is typed. For instance, if any ‘control’ keyword or key phrase is outgoing on the smartphones texting transmission bar, the application may sense similarity of the outgoing text to the ‘control’ key text. Using natural language processing or machine learning algorithms a ‘match’ or ‘no match’ may result by the application 104. If no match is determined, no action is taken. If a ‘match’ is determined, the ‘match’ may activate the smartphone camera to begin capturing a photo burst of the person, as they are typing the key word text. The app may then confirm the subject's identity using facial analytics. If the identity is confirmed as the subject, the application may capture facial images as the text is typed. The application 104 may capture both the keyword and the synchronous facials and compare the live database words/photos to the previously established control keyword/facial database. The app may then store each ‘match’ as a quantifiable integer.

Preservation of the texting associated integer may occur while the outgoing text is deleted. Likewise preservation of the fiducial facial grid points may occur, while the patient's picture is deleted. After each keyword and facial is captured throughout the day and stored in the corresponding integer is stored in the appropriate database the app may sum up the integers. A positive or negative integer may result.

In both instances of texting database or facial analytics, application 104 has used the same structure. Initially a baseline setup database is created using patient directed input. For both text and facial analytics, the setup database acts as a sensor to identify key outgoing texts or images. The setup data also serves as a trigger to allow the application to store, score and move the key data through the algorithm. In both embodiments the stored and scored key data is then associated with its previously embedded, randomized integers. If the integers are considered critical based upon the comparison to a content communication is generated in response. In both instances the application 104 may continue to retain and sum up the valued integers resulting in a set of final coordinates. These final coordinates are created using a patient established mental health/addiction database that is randomized and anonymized before installation. This patient associated data is sensed using a lexical resource, and moved through databases that convert live patient texts to a random number. In both embodiments of texting or facial analytics, the random number is then processed through sentiment analysis and subjectivity analysis enabling the AI to ‘learn’ new mental health/addictive textual words and phrases.

In an embodiment, the mobile device 102 is embodied as a dedicated mental health and addiction smartphone. In such a case, the mobile device 102 dedicated processing power for facial analytics and powering the artificial intelligence and text mining specifications needed for the application 104 to operate.

The control database may be set up with the involvement of a MH/A paraprofessional or the patient themselves prior to usage of the application 104. The outgoing text is a set of key words or phrases which serve as triggers for the mental health and addiction issue. Some of these text messages are consistent with a situation of recovery and some with relapse. Examples of text messaging used in the control database embodiment could include, but are not limited to appreciative stems, task stems, social support, avoidance texts/textual ‘triggers’, self-reappraisal phrases. In an embodiment, the patient may possess key texting words or texting triggers that have significance especially to them. Typically, these are unique people, places and things associated with the patients mental health and addiction issues. Outgoing examples of text stems used by the application 104 are provided by the table below:

| Stems | Patient Textual Phrase |
|---|---|
| Appreciative stems | “I am feeling really strong about my |
| | addiction today’ |
| Task stem | ‘I plan to attend an AA meeting tonight, what |
| | about you?’ |
| Social support stem | ‘Joe (the patients sponsor) do you mind if I |
| | call you |
| Avoidance trigger stem | ‘I passed by the store where I get my beer |
| | just now, TEMPTED!!’ |
| Cognitive reappraisal stem | ‘I completed step 4 today feeling strong.’ |

This step of setting up an outgoing text database may involve a professional obtaining key triggers from various sources. These sources could include, but not limited to, a substance use history, collateral sources like spouse, friends, old hospital records, parole officers information, court documents, professional resource network (PRN), a patients verbalized review of a ‘typical day’ involving recurrence or mental health triggers, as well as a historical record of collecting the old text for a two-month period.

The step of establishing a score for the corresponding outgoing text database may involve the patient and/or the therapist assigning a positive or negative value to the text or phrase. This may be done as a function of recovery and good mental health/addiction triggers have a ‘+’ valued integer assigned, and a relapse or bad mental health/addiction trigger is associated with a ‘−’ valued integer.

The application 104 may run all the database words and associated numbers through a randomizer. In contrast to the prior art, this step may be done before the application 104 is active. The application 104 may numerically randomize every control text to substitute a series of anonymized numbers for people, places and things found in the control database. This may enable the patients triggers to be used as a random number. The application 104 may thereafter upload the randomized numbers and words in the database.

The application 104 may establish the facial database which may be associated with the outgoing text database. In this database the key outgoing text may serve as a trigger for the camera to turn itself on, confirm identity via facial fiducial analysis, and capture a burst of images of the subject as they are texting. If there is considered a 75% match of facial fiducial points then that image may be sensed, scored and stored as an associated facial data. The patient main obtain baseline facial images or selfies involving the standard 6 emotions. Also obtained may be a set of emotions established in the context of mental health/addictive recovery or relapse. Standard mental health/addiction emotions may need to be established as well.

To obtain these standard facial emotions, the therapist may have the patient in a photo studio where they may be able to obtain the control facial datapoints corresponding to remission, recurrence, positive mental health, and negative mental health. One example of obtaining addiction facial action units could include the therapist may have the patient sitting in front of a camera and a monitor where they may be shown pictures of a key substance of use (cocaine, heroin with a needle, alcohol). Regarding the generation of mental health action units (MHAU's), an example of such ‘high stress’ could be pictures of war crime atrocities, cruelty to animals, difficult emotional events in the subject's life (except in PTSD).

As these stimuli are shown to the individual, the facial images of the subject may be captured and analyzed for high stress MHAU's. The resulting high stress MHAU's may be collated into the database. A similar process may be used for low stress MHAU's. (using happy pictures of family births/birthday parties/reunions/favorite pets.) (‘happy’ AU 12,25) These facial fiducial points may be stored in the database to be used as a ‘trigger’ to generate a facial action unit (FAU) score.

Furthermore, associated smell or sounds can be layered upon the basic visual stimulus to create a compound stimulated addiction or mental health environment. Within this mental health/addictive environment pictures may be taken of the subject's face as they are exposed the simulation environment. These facial pictures of the controlled mental health moment may be analyzed by state-of-the-art facial recognition software. Using state-of-the-art facial analysis software the application can analyze 30 frames/sec. This analysis may result in vectors/data points that are not just unique to the patient, but subordinate to the patient's addictive/mental health moment.

In another embodiment this key word data and scores may be sent on to the Blockchain for storage. It is acknowledged this is parceled outgoing text data that has already been sent out over the internet, unsecured. Storage in a blockchain format enables patient data to be encrypted within the blockchain.

In the foregoing and following description, numerous specific details, examples, and scenarios are set forth in order to provide a more thorough understanding of the present disclosure. It may be appreciated, however, that embodiments of the disclosure may be practiced without such specific details. Further, such examples and scenarios are provided for illustration only, and are not intended to limit the disclosure in any way. Those of ordinary skill in the art, with the included descriptions, should be able to implement appropriate functionality without undue experimentation.

References in the specification to “an embodiment,” etc., indicate that the embodiment described may include a particular feature, structure, or characteristic. Such phrases are not necessarily referring to the same embodiment. Further, when a particular feature, structure, or characteristic is described in connection with an embodiment, it is believed to be within the knowledge of one skilled in the art to effect such feature, structure, or characteristic in connection with other embodiments whether or not explicitly indicated.

Embodiments in accordance with the disclosure may be implemented in hardware, firmware, software, or any combination thereof. Embodiments may also be implemented as instructions stored using one or more machine-readable media which may be read and executed by one or more processors. A machine-readable medium may include any suitable form of volatile or non-volatile memory.

Modules, data structures, and the like defined herein are defined as such for ease of discussion, and are not intended to imply that any specific implementation details are required. For example, any of the described modules and/or data structures may be combined or divided in sub-modules, sub-processes or other units of computer code or data as may be required by a particular design or implementation of the computing device.

In the drawings, specific arrangements or orderings of elements may be shown for ease of description. However, the specific ordering or arrangement of such elements is not meant to imply that a particular order or sequence of processing, or separation of processes, is required in all embodiments. In general, schematic elements used to represent instruction blocks or modules may be implemented using any suitable form of machine-readable instruction, and each such instruction may be implemented using any suitable programming language, library, application programming interface (API), and/or other software development tools or frameworks. Similarly, schematic elements used to represent data or information may be implemented using any suitable electronic arrangement or data structure. Further, some connections, relationships, or associations between elements may be simplified or not shown in the drawings so as not to obscure the disclosure.

This disclosure is considered to be exemplary and not restrictive. In character, and all changes and modifications that come within the spirit of the disclosure are desired to be protected. While particular aspects and embodiments are disclosed herein, other aspects and embodiments may be apparent to those skilled in the art in view of the foregoing teaching.

### EXAMPLES

Illustrative examples of the technologies disclosed herein are provided below. An embodiment of the technologies may include any one or more, and any combination of, the examples described below.

Example 1 includes a device and method of a patient controlled, network independent mobile device for patient health assessment. The device is a “stand-alone” network-independent device having one or more patient-based and patient-associated, database. One such database serves as a control database for outgoing data. The data serves as a trigger or actuator of the application, pertaining to patient-associated data, occurring in real-time and real-world situations. The texting data is gathered and processed by an application executing on the mobile device. Another database a control database that is a patient-derived database serving as a preparation center for application between text data and control data. Control data serves as the measurable data placed within the database. The control data is associated with biological, health or, physiological data associated with the patient. Prior to insertion into the application this data is randomized to result in numerical anonymization of the data. This live, controlled and randomized data will be generally gathered, prepared and analyzed by the application. The application uses concepts described by affective computing and specifically implemented using natural language programing (NLP) and text mining (TM) techniques. Application of some combination of NLP and TM will be incorporated in the application to derive at a definable state of situational and contextual polarity for said entity. Using methods of NLP and machine learning algorithms, the application incorporates concepts of affective computing to achieve a level of artificial intelligence (AI). The AI will result in the incorporation of levels of consciousness using embedded text censors resulting in the ability of the application to learn from prior outgoing texts. This application uses data visualization in the form of a simple integer or user-friendly symbolism reflecting the output of said processes. This described data can also be placed in blockchain storage.

Example 2 includes the subject matter of Example 1, in which the mobile device has mobility characteristics.

Example 3 includes the subject matter of any of Examples 1 and 2, in which the patient is a person or an artificially controlled entity (e.g., a robot device).

Example 4 includes the subject matter of any of Examples 1-3, in which one of the databases stores historical data previously been transmitted from the patient source.

Example 5 includes the subject matter of any of Examples 1-4, in which one of the databases store patient-associated data characterized as data possessing some fraction of involuntary intention, including texting or facial biometrics are a result of a fraction of involuntary brain waves generated in the frontal cortex, posterior superior sulcus and the amygdala.

Example 6 includes the subject matter of any of Examples 1-5 in which one of the databases stores patient-associated and therapist-directed data, in which a therapist pertains to a professional or para-professional that treats a patient.

Example 7 includes the subject matter of any of Examples 1-6 in which the one of the databases is augmented using third-party software (e.g., WordNet) to expand the synonyms placed within the controlled database.

Example 8 includes the subject matter of any of Examples 1-7, in which the processed data is randomized prior to uploading into the database.

Example 9 includes the subject matter of any of Examples 1-8, in which one of the databases is embodied as a spreadsheet or comma separated value (CSV) file.

Example 10 includes the subject matter of any of Examples 1-9, in which control data is directed by manually constructed algorithms and databases.

Example 11 includes the subject matter of any of Examples 1-10, in which text data comprises patient-associated data present on a device outgoing communication status bar.

Example 12 includes the subject matter of any of Examples 1-11, in which live data comprises facial biometric data, in which the facial biometric data comprises data originally generated from the area of the posterior superior sulcus/amygdala or frontal cortex or resulting from facial biometric analysis.

Example 13 includes the subject matter of any of Examples 1-12, in which the control data is managed using a data management system (e.g., MongoDB, MySQL and Postgres).

Example 14 includes the subject matter of any of Examples 1-13, in which control data is prepared or processed through a numerical randomizer prior to upload in one of the databases.

Example 15 includes the subject matter of any of Examples 1-14, in which the prepared data is processed via techniques of word frequency, collocation and concordance, tokenization, stemming, lemmatization, word stop removal, semantic class tags, named entities, techniques of match extraction patterns, part-of-speech tagging. Abney stemmers, parse trees both constituency and dependency and clue finders.

Example 16 includes the subject matter of any of Examples 1-15, in which data analysis is performed using subjectivity and sentiment analysis techniques, including Naïve Bayes classifier, lexical and contextual features, rules-based classifiers, using conditional Random Field Sequence tagging models with extraction pattern learning to identify expression and polarity.

Example 17 includes the subject matter of any of Examples 1-16 in which the data analysis is used to update one or more of the databases.

Example 18 includes the subject matter of any of Examples 1-17, in which where Artificial Intelligence is used to determine a situational or contextual state of the user.

Example 19 includes the subject matter of any of Examples 1-18, in which where the data analysis results in the application sending positive reinforcement content to the user to serve as an ecological momentary assessment and a reinforcement or extinction of the impending situation.

Example 20 includes the subject matter of any of Examples 1-19, in which the content is a personalized data file, movie file, text file, or voice file.

Example 21 includes the subject matter of any of Examples 1-20, in which the application carries out the aforementioned functionalities independent of a network connection.

Example 22 includes the subject matter of any of Examples 1-21, in which the application carries out the aforementioned functionalities independent of a prescription prepared for the user.

Example 23 includes the subject matter of any of Examples 1-22, in which the application operates independent of a remote server.

Example 24 includes the subject matter of any of Examples 1-23, in which data analysis of the application results in an integer or human-readable symbol (e.g., an emoji) or combination of both.

Example 25 includes the subject matter of any of Examples 1-24, in which the randomized data integers maybe easily processed through the original randomizer for decryption if desired.

Example 26 includes the subject matter of any of Examples 1-25, in which the selected data is stored in blockchain format.

Example 27 includes the subject matter of any of Examples 1-26, in which the application controls an ‘on-off’ status of the communication device.

Example 28 includes the subject matter of any of Examples 1-27, in which the device is mobile.

Example 29 includes the subject matter of any of Examples 1-28, in which one of the databases stores patient-associated historical data.

Example 30 includes the subject matter of any of Examples 1-29, in which a patient is defined as a person or an artificially control entity (e.g., a robot).

Example 31 includes the subject matter of any of Examples 1-30, in which one of the databases stores patient-associated historical data.

Example 32 includes the subject matter of any of Examples 1-31, in which one of the databases stores patient-associated data.

Example 33 includes the subject matter of any of Examples 1-32, in which one of the databases stores patient-generated and therapist-directed data.

Example 34 includes the subject matter of any of Examples 1-33, in which processed data is randomized prior to upload into the database.

Example 35 includes the subject matter of any of Examples 1-34, in which one of the databases is embodied into a spreadsheet or a CSV file.

Example 36 includes the subject matter of any of Examples 1-35, in which one of the databases is augmented using WordNet to expand the synonyms placed within the database.

Example 37 includes the subject matter of any of Examples 1-36, in which the control data is directed by manually constructed algorithms and databases.

Example 38 includes the subject matter of any of Examples 1-37, in which the control data is text data.

Example 39 includes the subject matter of any of Examples 1-38 in which the control data is facial biometric data.

Example 40 includes the subject matter of any of Examples 1-39 in which the control data is managed in a data management system, such as MongoDB, MySQL and Postgres.

Example 41 includes the subject matter of any of Examples 1-40 in which the control data is processed through a numerical randomizer prior to upload in the database.

Example 42 includes the subject matter of any of Examples 1-41 in which the prepared data is processed via techniques of word frequency, collocation and concordance, tokenization, stemming, lemmatization, word stop removal, semantic class tags, named entities, techniques of match extraction patterns, part-of-speech tagging, Abney stemmers, parse trees both constituency and dependency and clue finders.

Example 43 includes the subject matter of any of Examples 1-42, in which the data analysis is performed using subjectivity and sentiment analysis techniques including Naïve Bayes classifier, Support Vector Machines, (SVM), deep learning algorithms, lexical and contextual features, rules-based classifiers, using conditional Random Field Sequence tagging models with extraction pattern learning to find expression and polarity.

Example 44 includes the subject matter of any of Examples 1-43, in which the data analysis is used to update the one or more databases.

Example 45 includes the subject matter of any of Examples 1-44, in which the application determines situational/contextual state using Artificial Intelligence techniques.

Example 46 includes the subject matter of any of Examples 1-45, in which the data analysis results in the application transmitting content to the user to act as an ecological momentary assessment and serve as reinforcement or extinction of the impending situation.

Example 47 includes the subject matter of any of Examples 1-46, in which the content comprises a personalized data file, movie file, and voice file.

Example 48 includes the subject matter of any of Examples 1-47, in which the application operators independent of a network.

Example 49 includes the subject matter of any of Examples 1-48, in which the application operates independently of a user obtaining a treatment prescription.

Example 50 includes the subject matter of any of Examples 1-49, in which the application operates independently from a remote backend server.

Example 51 includes the subject matter of any of Examples 1-50, in which the result of the data analysis is an integer or a human-readable emoji or combination of both.

Example 52 includes the subject matter of any of Examples 1-51, in which a final ‘randomized’ data integers is further processed through the original randomizer for unencryption.

Example 53 includes the subject matter of any of Examples 1-52, in which selected components are stored in blockchain format.

Example 54 includes the subject matter of any of Examples 1-53, in which the application uses facial biometric data generated from a therapist and patient-associated source data is internally of a patient database of a device.

Example 55 includes the subject matter of any of Examples 1-54, in which the facial biometric datapoints generated in an environment resulting in facial co-ordinates for the purpose of constructing a subjective database to be used for physiological and mental assessment.

Example 56 includes the subject matter of any of Examples 1-55, in which the facial database stores historical facial datapoints and co-ordinates.

Example 57 includes the subject matter of any of Examples 1-56, in which the facial database stores patient associated data.

Example 58 includes the subject matter of any of Examples 1-57, in which the facial database stores patient-generated and therapist-directed data.

Example 59 includes the subject matter of any of Examples 1-58, in which the facial biometric coordinates are stored in a spreadsheet or CSV file.

Example 60 includes the subject matter of any of Examples 1-59, in which the facial live data is directed using manually constructed algorithms.

Example 61 includes the subject matter of any of Examples 1-60, in which the facial data, i.e. facial fiducial points, are processed through a numerical randomizer prior to upload in a database with resultant facial data deleted.

Example 62 includes the subject matter of any of Examples 1-61, in which the facial fiducial points are converted to textual sentiments.

Example 63 includes the subject matter of any of Examples 1-62, in which the algorithm selects facial points and corresponding emotions to prepare and process the emotions for downstream data analysis using techniques of word frequency, collocation and concordance, tokenization, stemming, lemmatization, word stop removal, semantic class tags, named entities, techniques of match extraction patterns, part-of-speech tagging, Abney stemmers, parse trees both constituency and dependency, and clue finders.

Example 64 includes the subject matter of any of Examples 1-63, in which the The device of claim 63 where the converted Facial ‘emotions’ having been ‘prepared’ are analyzed using subjectivity and sentiment analysis techniques as described above utilizing techniques such as: Naïve Bayes classifier, lexical and contextual features, rules-based classifiers, using conditional Random Field Sequence tagging models with extraction pattern learning to find expression and polarity of the converted facial co-ordinates.

Example 65 includes the subject matter of any of Examples 1-64, in which the facial data analysis results in an upgradable facial emotional control corresponding to a complex facial emotion.

Example 66 includes the subject matter of any of Examples 1-65, in which machine learning is used to determine a facial situational and contextual state.

Example 67 includes the subject matter of any of Examples 1-66, in which facial data analysis results the application transmitting a communication with content to provide an ecological momentary assessment which serves as reinforcement or extinction of the impending situation.

Example 68 includes the subject matter of any of Examples 1-67 in which the Facial text to self is a personalized data file, video file, text file, or a voice file.

Example 69 includes the subject matter of any of Examples 1-68, in which the application operates independent of a network.

Example 70 includes the subject matter of any of Examples 1-69, in which the application operates independent of an ongoing prescription of the user.

Example 71 includes the subject matter of any of Examples 1-70, in which the application operates independent from a remote server.

Example 72 includes the subject matter of any of Examples 1-71, in which the facial data analysis outputs a integer or human-readable symbol or combination of both.

Example 73 includes the subject matter of any of Examples 1-72, in which the resulting randomized data integers are processed through the original randomizer for decryption back to the key text.

Example 74 includes the subject matter of any of Examples 1-73, in which the selected data is stored in blockchain format.

Example 75 includes a method and a device involving a patient-controlled, network independent mobile device for patient health assessment executing an application. A method of using a stand-alone network independent device includes one or more patient-based and patient-associated databases. One such database includes a control database. One database is a control database storing outgoing data. The data serves as a trigger or actuator of the application. The nature of this data is demonstrated to be patient associated data, occurring in real-time and real-world situations. The texting data is gathered by the app and processed. A method where another database is a patient-derived database serving as a preparation center for the application. The preparation occurs between text data and the control data. Controlled data is gathered as patient generated and serves as the measurable data placed within the control database. Further, the controlled data is associated with a patients biological, health or physiological data. Prior to insertion into the application this data is randomized to result in numerical anonymization of the data. Live, controlled and randomized data will be generally gathered, prepared, and analyzed by the application. The application uses natural language programing (NLP) and text mining (TM) techniques to derive at a definable state of situational and contextual polarity for said entity. The described application will incorporate concepts of affective computing to achieve a level of artificial intelligence (AI). The AI techniques will levels of consciousness using embedded text censors resulting in the ability of the application to learn from prior outgoing texts. This application will claim a method of data visualization in the form of a simple integer or user-friendly symbolism reflecting the output of said processes. The described data may be placed in blockchain storage.

Example 76 includes the subject matter of Example 75, in which the device is functionally dependent on the application.

Example 77 includes the subject matter of any of Examples 75 and 76, in which NLP methods are integrated into previously described patient associated databases.

Example 78 includes the subject matter of any of Examples 75-77, in which the device has mobility.

Example 79 includes the subject matter of any of Examples 75-78, in which patient is defined as a person or an artificially controlled entity.

Example 80 includes the subject matter of any of Examples 75-79, in which NLP techniques are performed on patient-associated data contained in one or more databases for patient assessment.

Example 81 includes the subject matter of any of Examples 75-80, in which insertion of the updated patient-associated texts found within the databases is manually or automatically performed, and in which he update insertion results from natural language processing and is automatic, and in which these ‘software updates’ into the device occur with or independent of a network connection.

Example 82 includes the subject matter of any of Examples 75-81, in which final data integer are sent over the network to a healthcare provider with or without an encryption step.

Example 83 includes the subject matter of any of Examples 75-82, in which data associated with the application is sent for storage using blockchain techniques.

Example 84 includes the subject matter of any of Examples 75-83, in which patient-associated facial fiducial points are captured independent of a user prescription and used the outgoing patient associated transmission data, in which the facial fiducial points are used to sense, process and compare outgoing facial fiducial points to the stored facial fiducial points found in the aforementioned databases.

Example 85 includes the subject matter of any of Examples 75-84, in which the patient-associated facial fiducial points are captured and the use of texting in a database combined with or without facial analytics is used for the assessment of mental health and addiction, in which, prior to installing the application, a patient in a therapist photo studio exposes the patient to a camera taking a number of pictures involving a number of predictable and controlled environments that reminds them of the mental health issue or an addictive moment (also referred to herein as therapist- or patient-directed self-images, or selfies), in which controlled patient-associated selfies comprise pictures of the individual in an environment simulating an addictive environment or mental health triggers, in which examples of this controlled environment might include pictures of a line of cocaine, taking heroin or a picture of the patient's favorite bar, in which if there is an associated smell or sounds that can be layered upon the basic visual stimulus, in which within this mental health/addictive environment pictures will be taken of the subject's face as they are exposed the “simulation” environment, in which facial pictures of the controlled mental health moment are analyzed by using facial recognition techniques, in which this analysis is further evaluated to discover mental health or addictive action units (AU) that are uniquely associated with the subject and their addiction, in which the AU's are collated into a patient-associated fiducial facial database, in which the studio will also capture facial fiducial points associated with sadness and stress will be shown to the individual, in which an example of such high stress would be pictures of war crimes atrocities, cruelty to animals, difficult emotional events in the subject's life such as consequences from the recent COVID-19 viral pandemic, in which, as this stimulus is shown to the individual, the facial images of the subject will be captured and analyzed for unique mental health high stress AUs, in which the resulting patient associated mental health high stress AU's are collated, in which this mental health and addiction-controlled environment facial action units are processed through state-of-the-art facial recognition software, in which a patient assisted controlled facial fiducial points are generated without recording the patient's photo, in which patient's picture is destroyed but the fiducial points are retained, in which these AU points are stored in the database, in which the application will confirms the subject with a facial point analysis, in which if there is no facial match to the patient the camera will not be activated, in which if a patient to photo match is confirmed, the camera will be activated, in which the incoming and outgoing keyword texts will be verified, and a photo burst obtained as the subject is reading or composing keyword texts, in which a facial video or photo burst will be obtained while the subject is reading or composing the texts, in which the selfie and text are analyzed using artificial intelligence and an integer is generated, in which if the natural language processing determines a “no match,” a zero integer is given to the patient associated fiducial points, in which if the processing method results in a “match” reflecting a positive mental health moment or addictive remission, a positive integer is stored with the text, in which a negative integer results from the converse.

Example 86 includes the subject matter of any of Examples 75-85, in which a timed response is generated for transmitting a communication to the user, in which this timed response maybe immediate and/or delayed, in which an internal communication from the device without sending a response is used independent of a network connection, in which the communication uses a natural language processing to prepare the content of the response as well as machine learning algorithms to determine the appropriate response, in which the data is used a trigger, in which the sensed patient-initiated data is a match with a content database, then the patient mobile device response is generated, in which if the outgoing text is a match an immediate special patient-initiated data file will be sent from the patient to the patient's mobile phone using this method, in which a method is described where the generated response will be data such as words, phrases, pictures or voice files, in which the device triggers and sends customizable, timely TTS transmission interrupting the addiction or mental health moment is proposed.

Example 87 includes the subject matter of any of Examples 75-86, in which a trigger actuates a mobile communication device using a database to enable the intervention of a person with correctable issues; in which patient-associated data base are constructable, in which the described database is used as a control platform.

Example 88 includes the subject matter of any of Examples 75-87, in which the described data points include text data, voice files, pictures, video files or GPS data points as a trigger to initiate a content communication.

Example 89 includes the subject matter of any of Examples 75-88, in which the content communication initiates an action to copy and send the outgoing data file for analysis.

Example 90 includes the subject matter of any of Examples 75-89, in which the sensed and transmitted data is analyzed using a straightforward database comparison or NLP and/or ML.

Example 91 includes the subject matter of any of Examples 75-90, in which NLP and ML algorithms are used to extract and determine a “match” or a “no match” between the outgoing data files and the stored database files.

Example 92 includes the subject matter of any of Examples 75-91, in which a “match” or “no match”′ threshold is determined, in which if a match occurs, then the device and or application gathers the matched data, in which Once the outgoing matched data has been gathered, action to process and prepare the data is taken, in which the matched data is then analyzed to determine an integer as well as a positive or negative value of the integer associated with the outgoing data, in which the resulting integer or symbolic representation is visualized.

What is claimed is:

1. A method comprising:
detecting, by a mobile device, a communication from a user;
determining, by the mobile device, present attributes associated with the mobile device;
determining, by the mobile device and as a function of the communication and the present attributes, a likelihood that a relapse or a recovery event associated with the user is to occur; and
in response to determining that a relapse or recovery event is to occur, transmitting a content communication responsive to the relapse or recovery event to the user.

2. The method of claim 1, wherein the communication is one of a text or image message, a voice communication, and a video communication.

3. The method of claim 1, wherein the content communication is one of a text message, a voice communication, and a video communication.

4. The method of claim 1, wherein the present attributes comprises at least one of location data and a time and date associated with the communication from the user.

5. The method of claim 1, wherein the content communication is transmitted to the user independent of a network communication.

6. The method of claim 1, wherein the content communication is transmitted to the user independent of a backend server.

7. The method of claim 1, wherein determining the likelihood that a relapse or a recovery event is to occur comprises evaluating the communication using natural language processing techniques.

8. A mobile device, comprising:
one or more processors; and
a memory storing program code, which, when executed by the one or more processors, causes the mobile device to:
detect a communication from the user;
determine present attributes associated with the mobile device;
determine, as a function of the communication and the present attributes, a likelihood that a relapse or a recovery event associated with the user is to occur; and
in response to determining that a relapse or recovery event is to occur, transmit a content communication responsive to the relapse or recovery event to the user.

9. The mobile device of claim 8, wherein the communication is one of a text or image message, a voice communication, and a video communication.

10. The mobile device of claim 8, wherein the content communication is one of a text message, a voice communication, and a video communication.

11. The mobile device of claim 8, wherein the present attributes comprises at least one of location data and a time and date associated with the communication from the user.

12. The mobile device of claim 8, wherein the content communication is transmitted to the user independent of a network communication.

13. The mobile device of claim 8, wherein the content communication is transmitted to the user independent of a backend server.

14. The mobile device of claim 8, wherein to determine the likelihood that a relapse or a recovery event is to occur comprises evaluating the communication using natural language processing techniques.

15. One or more machine-readable storage media, which, when executed on a mobile device, causes the mobile device to:
detect a communication from the user;
determine present attributes associated with the mobile device;
determine, as a function of the communication and the present attributes, a likelihood that a relapse or a recovery event associated with the user is to occur; and
in response to determining that a relapse or recovery event is to occur, transmit a content communication responsive to the relapse or recovery event to the user.

16. The one or more machine-readable storage media of claim 15, wherein the communication is one of a text or image message, a voice communication, and a video communication.

17. The one or more machine-readable storage media of claim 15, wherein the content communication is one of a text message, a voice communication, and a video communication.

18. The one or more machine-readable storage media of claim 15, wherein the present attributes comprises at least one of location data and a time and date associated with the communication from the user.

19. The one or more machine-readable storage media of claim 15, wherein the content communication is transmitted to the user independent of a network communication or a backend server.

20. The one or more machine-readable storage media of claim 15, wherein to determine the likelihood that a relapse or a recovery event is to occur comprises evaluating the communication using natural language processing techniques.
